(19) United States
(12) Patent Application Publication
(10) Pub. No.: US 2021/0059811 A1
(43) Pub. Date: Mar. 4, 2021

(54) INJECTOR SYSTEM FOR INTRAOCULAR LENSES

(71) Applicant: MEDICONTUR MEDICAL ENGINEERING LTD., Zsambek (HU)

(72) Inventors: Peter HANGYA, Erdoalja (HU); Laszlo Ferenc KONTUR, Budapest (HU)

(21) Appl. No.: 16/958,317

(22) PCT Filed: Dec. 28, 2017
(86) PCT No.: PCT/HU2017/000053

§ 371 Date: Jun. 26, 2020

(51) Int. Cl. A61F 2/16

(52) U.S. Cl. CPC A61F 2/167; A61F 2/1691; A61F 2/1678; A61F 2/1675

(57) ABSTRACT

For a safe injection of intraocular lenses, IOL, (1) an injector is provided comprising a cartridge (5) with a nozzle tube (8), a lens case (6), an injector body (2), an injection plunger (4) and a loading pusher (3). The cartridge (5) comprises two winglets (12, 13) connected to each other providing an open and a close position for the cartridge (5), the inner surface of the winglets forming an inner space for receiving the IOL, and moving the IOL (1) during closing the cartridge (5) from a level of loading plane (16), where the axis of the inner space coincides with the axis of the loading pusher, to a level of injection plane (17), where the axis of the inner space coincides with the axis of the injection plunger. The lens case (6) is attached to the injector body behind the cartridge and in an advantageous embodiments has an upper position and a lower position for preloaded IOL (1).

### TECHNICAL FIELD

The present invention relates to a surgical injector system, more particularly, to injectors with lens cases for non-preloaded as well as for preloaded intraocular lenses (IOLs) and methods for operation thereof.

### BACKGROUND

An IOL is an artificial lens implanted in the eye, for example, as a replacement for the natural crystalline lens after cataract surgery or to alter the optical properties of an eye in which the natural lens remains. IOLs include an optic, and preferably at least one flexible fixation member or haptic which extends from the optic and becomes affixed in the eye to secure the lens in position. The optic normally includes an optically clear lens. Implantation of such IOLs into the eye involves making an incision in the eye. It is advantageous, to reduce trauma and accelerate healing, to have an incision size as small as possible. Modern IOLs are foldable so that the IOL can be inserted through a smaller incision into the eye. A variety of instruments have been proposed to aid in inserting such a foldable lens in the eye.

In the beginning the surgeon simply used surgical forceps having opposing blades which were used to grasp the folded IOL and insert it through the incision into the eye. While this method is practically not in use anymore, most surgeons are using more or less sophisticated IOL injectors offering more control to the surgeon when inserting the IOL into the eye. IOL injectors have recently been developed with reduced diameter nozzles which allow for a much smaller incision to be made in the cornea than is possible using forceps only. Smaller incision sizes (e. g., less than about 3 mm) are preferred over larger incisions (e.g., about 3.2 to 5+mm) since smaller incisions have been attributed to reduced post-surgical healing time and complications such as induced astigmatism.

Since IOLs are very small and delicate articles of manufacture, great care must be taken in their handling. In order for the IOL to fit through the smaller incisions, they need to be folded and/or compressed prior to entering the eye wherein they will assume their original unfolded/uncompressed shape. The IOL injector device must therefore be designed in such a way as to permit the easy passage of the IOL through the device and into the eye, yet at the same time not damage the delicate IOL in any way. Should the IOL be damaged during delivery into the eye, the surgeon will most likely need to extract the damaged IOL from the eye and replace it with a new IOL, a highly undesirable surgical outcome.

Several types of injector systems are available today and can be clearly distinguished and separated regarding the loading of the IOL. There are mainly two types of non-preloaded injector systems: Most common are the winglet type cartridge injectors where the unfolded IOL has to be loaded from the side, being prefolded by the closing winglets and then pushed forward through the cartridge nozzle by a plunger with a tip made from a soft material. This generic type of non-preloaded injector is the most universal type, usable for many kind of IOLs.

Disadvantage of injectors with non-preloaded winglet-type cartridge is the need to insert the IOL into the winglets by forceps requiring skill and experience of the user in order to avoid the risk of mishandling and/or damaging the lens during loading and closing the cartridge.

The other type of non-preloaded injector system has a fix-type cartridge that has to be loaded by the unfolded IOL from the back, where the lens is folded simply by being pushed through a narrowing cartridge nozzle by a pushing rod from hard material. These injector systems usually work only with hydrophobic IOLs and due to the lack of pre-folding of the IOL, the size of the nozzle tubes is larger. Patent specification WO1994007436 describes a non-preloaded winglet-type injector system with a pushing rod for hydrophobic IOLs

Besides the non-preloaded injector systems there are different so-called preloaded injector systems. In case of some preloaded injector systems the hydrophobic IOL is placed into the injector during the assembly process of the injector and the IOL and the injector are packed, sterilized, stored and shipped together. These are the single-phase, truly preloaded IOL/injector systems.

Patent specifications U.S. Pat. No. 7,156,854 and WO2007080869 both describe such a single-phase, preloaded injector system with a hard pushing rod for hydrophobic IOLs.

In case of hydrophilic preloaded IOL/injector systems the IOL is usually preloaded in a special lens case or container, packed, sterilized, stored and shipped separately from the injector. The reason for this is the different sterilization process needed for the dry-packed injector (ethylenoxide) and for the hydrated IOL (steam). Before injection the lens case/cartridge with the preloaded hydrophilic IOL has to be put on the injector, thus creating one injection system with a preloaded IOL. Some of these systems are winglet type, some of them backloaded type injectors.

Patent specification WO2007027499 describes an injector system for hydrophilic IOLs, enabling the user to load the IOL into the injector system without touching the IOL.

Patent specification WO2007078602 illustrates a folding arrangement for non-preloaded injectors in which small sized hydrophilic IOLs can be folded prior to injection by a rotational movement. However, the IOL is primarily stored in a vial before it has to be loaded into the cartridge.

Both arrangements are suitable for hydrophilic IOLs only and can be ruled out for hydrophobic IOLs.

Looking at these different kinds of injector systems, all of them with their own limitations, our target with this invention is to create a universal intraocular lens injector system that can be used as a preloaded or non-preloaded system, for hydrophobic or hydrophilic lenses. In all cases we want to use the same basic injector setup with the same type of cartridge suitable for safely loading and injecting preloaded and non-preloaded IOLs, from hydrophobic or hydrophilic materials.

### SUMMARY

We realized that combining the winglet-type cartridge with a controlled loading mechanism in a single device can result in an advantageous construction suitable for both non-preloaded and preloaded IOLs. This combination can be carried out by separating the levels of loading and injection planes.

In case of non-preloaded IOLs, i.e. when the injector and the IOL are shipped separately, this injector system is suitable for most types of foldable IOLs, both hydrophilic and hydrophobic. Its lens case allows a very simple and safe loading of the unfolded IOL minimizing the risk of any mishandling or damage of the IOL.

This injector system is also capable of receiving a closed lens case preloaded with a IOL, thus it is a 2-phase preloaded injection system for preloaded IOLs.

Finally, with the help of a modified lens case pre-assembled with the injector it can function as a single-phase preloaded IOL/injection system for hydrophobic IOLs.

In all cases the key element of the injector is the cartridge providing two different levels (i.e. planes) for the IOL. The loading of the IOL is carried out in the open position of the cartridge at an upper plane, i.e. at the level of the loading plane. During closing the cartridge, two operations are performed at the same time: on one hand the IOL is pre-folded and on the other hand the pre-folded IOL descends from the upper loading level to a lower injection level, which is aligned with the axis of the injection plunger.

The other key element of the invention is a lens case, sitting behind the cartridge, from where the loading of the IOL into the cartridge can be carried out. This lens case has different versions. One version that can be opened is sitting fixed behind the cartridge and has to be loaded with a non-preloaded IOL. Another version of this lens case is preloaded, separately sterilized and packed with a IOL and can be mounted in a single movement on the injector behind the cartridge. A third version of the lens case for preloaded hydrophobic IOLs is pre-mounted on the injector, having two positions: an upper storage/shipping position (i.e. level) and a lower loading position (i.e. level) where the lower loading level of the lens case is aligned with the upper loading level of the cartridge mentioned above. Such a lifting lens case is mounted on the injector in the upper position providing safe storage/shipping function. During the usage, the lifting lens case is moved to the lower position, to the loading level. This version of the lens case in cofunction with the cartridge creates three different functional levels (i.e. planes) for the IOL in the injector: a storage/shipping level, a loading level and an injection level.

Accordingly, the invention relates to an injector system for injecting foldable IOLs. The injector system comprises a cartridge with a nozzle tube, a lens case, an injector body, an injection plunger and a loading pusher. The injector body accommodates the loading pusher and the injection plunger the axis of which are parallel to each other and are accommodated in the injector body on a level of loading and on a level of injection, respectively. The cartridge comprises two winglets connected to each other by a hinge joint providing an open and a close position for the cartridge. In the open position of the cartridge, the inner surface of the winglets forms an inner space on the level of the loading plane, where the plane of the inner space is aligned (coincides) with the axis of the loading pusher. In the closed position of the cartridge, the inner surface of the winglets forms an inner space on the lower level of the injection axis, where the axis of the inner space coincides with the axis of the injection plunger. The lens case is attached to the injector body behind the cartridge. The inner surface of the lens case forms an inner space for the unfolded IOL.

In an advantageous embodiment, the lens case attached to the injector body has an upper position where the plane of the inner space of the lens case is above the level of loading, and a lower position where the plane of the inner space of the lens case is on the level of the loading plane, where the longitudinal axis of the inner space coincides with the axis of the loading pusher. In other words, in the embodiment for such a lifting lens case three levels are defined for the IOL in this embodiment: 1.) storage and shipping the IOL in the upper position of the lens case, 2.) loading of the IOL in the lower position of the lens case that is on the level of the loading plane and 3.) the level of the injection axis.

The invention also relates to a method of operating the injector. Three methods are developed, one for non-preloaded IOLs, one for IOLs preloaded in a separate lens case and one for hydrophobic preloaded IOL.

In case of injecting a non-preloaded IOL, the method comprises the steps of a.) injecting visco-elastic material into the open cartridge and into the open lens case; b.) placing the IOL into the lens case; c.) closing the lens case; d.) moving the IOL into the cartridge by pushing forward the loading pusher; e.) pulling back the loading pusher; f.) closing the cartridge; g.) injecting the IOL by pushing forward the injection plunger.

In case of injecting an IOL preloaded in a separate lens case, the method comprises the steps of a.) mounting the lens case preloaded with an IOL; b.) injecting visco-elastic material into the open cartridge and into the lens case through a hole; c.) moving the IOL into the cartridge by pushing forward the loading pusher; d.) pulling back the loading pusher; e.) closing the cartridge; f.) injecting the IOL by pushing forward the injection plunger.

In case of injecting a hydrophobic preloaded IOL, the method comprises the steps of a.) moving the lifting lens case from an upper position to a lower position and injecting visco-elastic material to the open cartridge and into the lens case through a hole; b.) moving the IOL into the cartridge by pushing forward the loading pusher; c.) pulling back the loading pusher; d.) closing the cartridge; e.) injecting the IOL by pushing forward the injection plunger.

This construction allows the IOL to be loaded, folded and injected by the injector in a safe way preventing mishandling and damage of the IOL.

### BRIEF DESCRIPTION OF THE DRAWINGS

For a more complete understanding of the invention, reference is made to the following detailed description of an embodiment taken in conjunction with the accompanying drawings wherein:

FIG. 1 shows the injector for non-preloaded IOL in disassembled state;

FIG. 2 shows the injector for non-preloaded IOL in assembled state;

FIG. 3 shows the injector for hydrophobic preloaded IOL in disassembled state;

FIG. 4 shows the injector for hydrophobic preloaded IOL in assembled state;

FIGS. 5 and 6 show the lens case for hydrophobic preloaded IOL in the injector by side view;

FIGS. 7 and 8 show the lens case for hydrophobic preloaded IOL in the injector by a sectional view;

FIGS. 9A, 9B and 9C show the structure and operation of cartridge by a cross sectional view;

FIG. 10 shows the step of injecting visco-elastic material to the cartridge and to the lens case of the non-preloaded IOL;

FIG. 11 shows the step of injecting visco-elastic material to the cartridge and to the lens case of the hydrophobic preloaded IOL;

FIG. 12 shows the step of placing the non-preloaded IOL in the lens case;

FIG. 13 illustrates the closing step of the lens case for non-preloaded IOL;

FIG. 14 shows the loading step when the non-preloaded IOL is moved to the cartridge;

FIG. 15 shows the same loading step from another view;

FIGS. 16 and 17 show the loading step for hydrophobic preloaded IOL;

FIG. 18 and FIG. 19 show the step of pulling back the loading pusher for non-preloaded and for hydrophobic preloaded IOL, respectively;

FIG. 20 shows the closing step of the cartridge for non-preloaded IOL;

FIG. 21 shows the closing step of the cartridge for hydrophobic preloaded IOL;

FIG. 22 shows the injection step for non-preloaded IOL;

FIG. 23 shows the injection step for hydrophobic preloaded IOL;

FIG. 24 shows the docking step of the IOL preloaded in a separate lens case;

FIG. 25 shows the removal step of the stopper from the lens case for preloaded IOL;

FIG. 26 shows injection step of visco-elastic material into the lens case for a preloaded IOL.

### DETAILED DESCRIPTION

In the following, for purpose of explanation and not limitation, specific details of an injector for IOLs are set forth, in order to provide a thorough understanding of the present invention. It will be apparent to one skilled in the art that the present invention may be practiced in other embodiments that depart from these specific details.

Embodiment of Injector for Non-Preloaded IOL

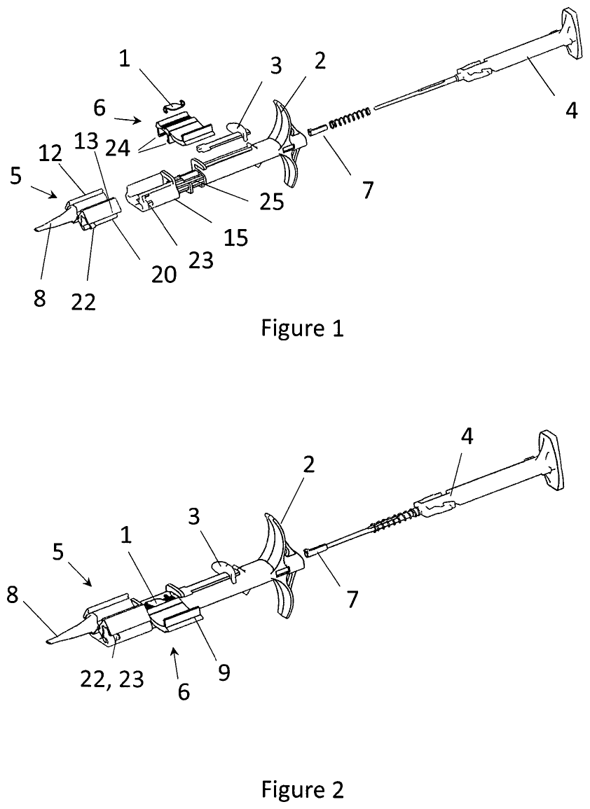

FIG. 1 shows the parts of an injector for injecting non-preloaded IOL 1. In this embodiment the injector comprises a cartridge 5 with a nozzle tube 8, a lens case 6, an injector body 2, an injection plunger 4 and a loading pusher 3. At the end of the injection plunger 4 a soft tip 7 is arranged. The injector body 2 accommodates the loading pusher 3 and the injection plunger 4. The axis of the two plungers are parallel to each other and both are accommodated in the injector body 2 in which they can reciprocate. The loading pusher 3 moves above the injection plunger 4 in a different plane. The cartridge 5 has two winglets 12, 13. A flange 20 of at least one winglet 13 is provided with a protrusion 22 fitting to a cavity 23 in the base 15 of the cartridge 5 in its closed position. The protrusion 22 fitting to a cavity 23 in the base 15 of the cartridge 5 in its closed position ensures that the cartridge 5 cannot be removed casually from the injector after closing the cartridge 5. In this embodiment, the bottom of the lens case 6 is provided with rims 24 and the injector body 2 is provided with grooves 25. The lens case 6 is attached to the injector body 2 behind the cartridge 5. The inner surface of the lens case 6 forms an inner space for the unfolded IOL 1. The IOL 1 is not part of the injector, it can be inserted to the lens case 6 of the injector just before the usage. The base 15 of the cartridge 5 is also part of the injector body 2.

FIG. 2 shows the assembled injector for non-preloaded IOL 1. All parts, i.e. the injector body 2, the cartridge 5 with the nozzle tube 8, the lens case 6 and the loading pusher 3 are put together except the injection plunger 4 with the soft tip 7. The IOL 1 is inserted in the lens case 6. The lens case 6 is connected to the injector body 2 by the rims 24 and the grooves 25 depicted in FIG. 1. In this assembled state, and the axis of inner space of the lens case 6 coincides with the axis of the loading pusher 3 when the rims 24 of the lens case 6 fit to the grooves 25 of the injector body 2. The lens case 6 is provided with a lid 9 hinging on the lens case 6, by which the lens case 6 can be closed after the IOL 1 was inserted in the inner space of the lens case 6. The protrusion 22 on the flange 20 of the winglet 13 fits to a cavity 23 in the base 15 of the cartridge 5. In further embodiments, more than one protrusion 22 on the flange 20 of the winglet 13 can be developed or further protrusions can be configures on the flange of the other winglet 12 too.

Embodiment of an Injector for Hydrophobic Preloaded IOLs

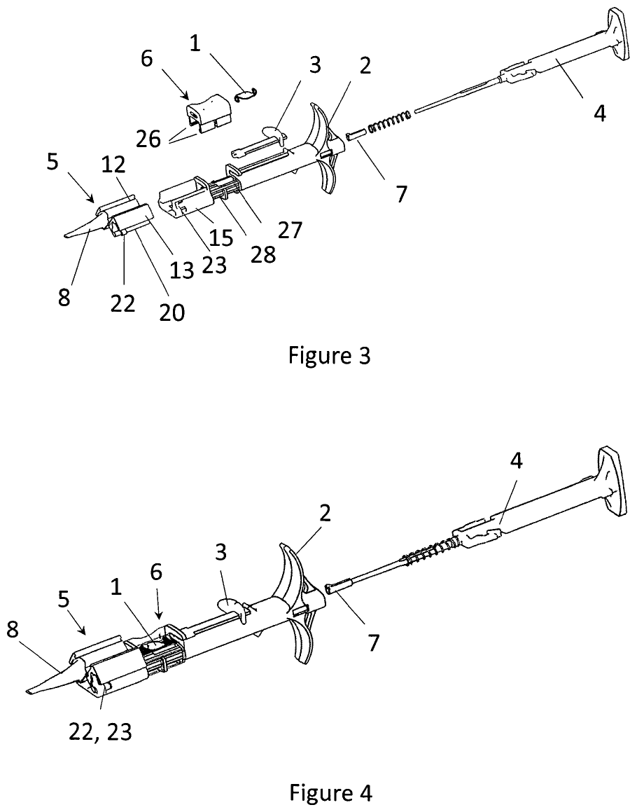

FIG. 3 shows the parts of an injector for injecting hydrophobic preloaded IOL 1. In this embodiment the injector also comprises a cartridge 5 with a nozzle tube 8, a lens case 6, an injector body 2, an injection plunger 4 and a loading pusher 3. At the end of the injection plunger 4 a soft tip 7 is arranged. The injector body 2 accommodates the loading pusher 3 and the injection plunger 4 similar to the embodiment in FIGS. 1 and 2. The axis of the two plungers are also parallel to each other and both are accommodated in the injector body 2 in which they can reciprocate. The loading pusher 3 moves above the injection plunger 4 in a different plane. The forms of cartridge 5 with the two winglets 12, 13, the flange 20, the protrusion 22 fitting to a cavity 23 in the base 15 of the cartridge 5 in its closed position can be developed identical as described in case of the previous embodiment. The base 15 of the cartridge 5 is also part of the injector body 2. The lens case 6 is attached to the injector body 2 behind the cartridge 5, the inner surface of the lens case 6 forms an inner space for the unfolded IOL 1. The difference to the injector of non-pre-loaded IOL, that the bottom of the lens case 6 is provided with rims 26 fitting to upper grooves 27 of the injector body 2 in an upper position of the lens case 6 and fitting to lower grooves 28 in a lower position of the lens case 6. So the lens case 6 can be attached to the injector body 2 in two positions. In the upper position, where the axis of the inner space of the lens case 6 is above the level of the loading plane and the IOL 1 cannot be loaded to the cartridge 5, and in the lower position where the axis of the inner space of the lens case 6 is on the level of the loading plane 16, where the axis of the inner space coincides with the axis of the loading pusher 3. In this embodiment, the IOL 1 is part of the injector, they are packed and shipped together.

FIG. 4 shows the assembled injector for preloaded IOL 1. All parts, i.e. the injector body 2, the cartridge 5 with the nozzle tube 8, the lens case 6 and the loading pusher 3 are put together except the injection plunger 4 with the soft tip 7. The protrusion 22 on the flange 20 of the winglet 13 fits to a cavity 23 in the base 15 of the cartridge 5 like in the non-preloaded case. The injector body 6 is shown transparently in this figure to reveal the position of the IOL 1 in the inner space of the lens case 6.

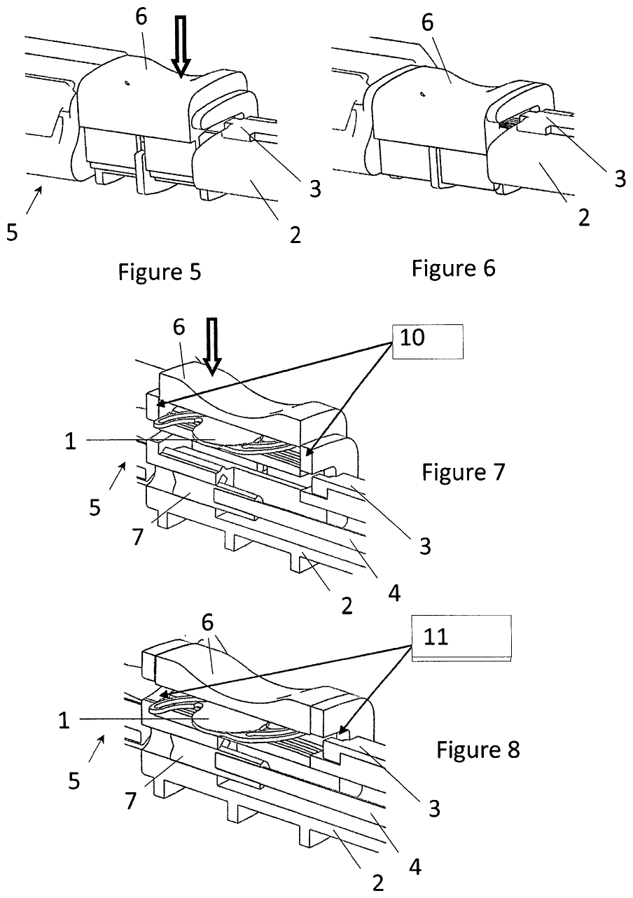

FIGS. 5, 6, 7 and 8 show the detailed structure and positions of the lens case 6 comprising on the IOL 1 and other parts of the injector. FIGS. 5 and 6 show the step of preparation for loading the preloaded IOL 1. By a side view. As it was mentioned, preloaded IOL 1 is shipped in the lens case 6 together with the injector. The lens case 6 is attached to the injector body 2 behind the cartridge 5 in an upper position as it is illustrated in FIG. 5. Then the lens case 6 is pushed down to a lower position, in the direction of the arrow. The lower position can be seen in FIG. 6. Separation of the upper and the lower position can prevent casual loading of the IOL 1.

In order to keep the IOL 1 in the lens case 6 during storage and shipment but let it loaded to the cartridge 5 before the usage, two gates 10 are provided on the injector body 2 at the both ends of the lens case 6 closing the both end of the inner space of the lens case 6 in its upper position as it is shown in FIG. 7. In the upper position of the lens case 6, gates 10 prevents IOL 1 getting out of the lens case 6. FIG. 7 is a sectional view that also illustrates the end of the loading pusher 3 and the end of the injection plunger 4 with the soft tip 7 reciprocating on different levels of planes.

When the lens case 6 is moved to the lower position, as it is depicted in the sectional view of FIG. 8, tunnels 11 at the both ends of the lens case 6 allow the loading pusher 3 to move the IOL 1 across the lens case 6 to the cartridge 5.

Embodiment of an Injector for Hydrophilic or Hydrophobic IOLs Preloaded in a Separate Lens Case

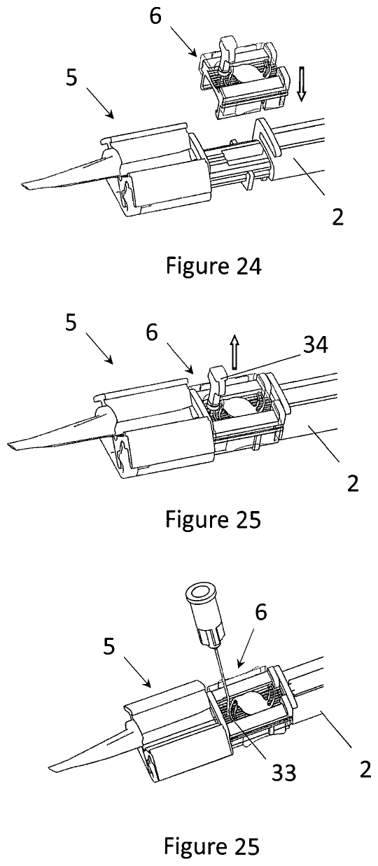

FIG. 24 shows the embodiment of the injection system with a lens case 6 for hydrophilic or hydrophobic preloaded IOL 1. The structure of this system is similar to the one described for hydrophobic preloaded IOL 1 however the lens case 6 is different. In this embodiment the lens case 6 has a lower portion capable for docking to the injector body 2. The arrow indicates the direction of the docking. After docking, the lens case 6 takes up a position for loading the IOL 1, i.e. the level of the IOL 1 coincides the level of the loading plane where the IOL 1 can be forwarded to the cartridge 5.

In FIG. 25, the lens case 6 is attached to the injector body 2. The lens case 6 comprises a stopper 34 that has to be removed before loading the IOL 1 to the cartridge 5. Removal of the stopper 34 is show by the arrow.

FIG. 26 shows that the lens case 6 comprises a hole 33. Through the hole 33 visco-elastic material can be injected into the lens case 6 to minimize the friction between the IOL and the inner spaces of the injector during loading and injecting the IOL.

Embodiment Details of the Cartridge for Non-Preloaded as Well as for Preloaded IOL

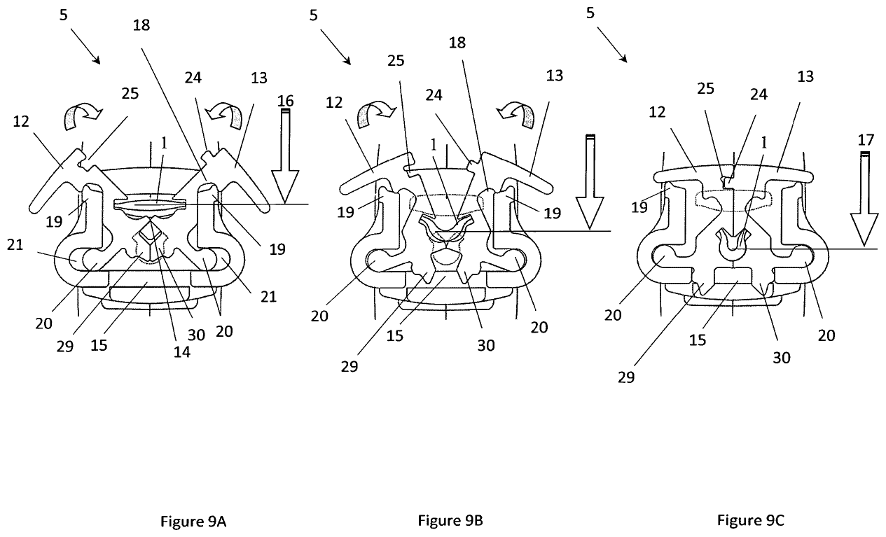

FIGS. 9A, 9B and 9C show the structure and operation of the cartridge 5 by cross sectional views. The cartridge 5 comprises two winglets 12, 13 connected to each other by a hinge joint 14 providing an open and a close position for the cartridge 5. The open position is illustrated in FIG. 9A, the close position in FIG. 9C. FIG. 9B shows an intermediate phase between the open position and the close position. In the open position of the cartridge 5, the inner surface of the winglets 12, 13 forms an inner space for the IOL 1 on the level of the loading plane 16, where the axis of the loading plane 16 coincides with the axis of the loading pusher. In the close position of the cartridge 5 the inner surface of the winglets 12, 13 forms an inner space for the IOL 1 on the level of the injection plane 17, where the axis of the inner space coincides with the axis of the injection plunger. The axis of the loading plane 16 as well as the axis of the injection plane 17 are perpendicular to the cross sections of the figures. The outer surface of the winglets 12, 13 at their upper parts are provided with recesses 18 fitting to flanges 19 of the base 15 of the cartridge 5 in the open position of the cartridge 5, and are provided with flanges 20 at their bottom parts fitting to recesses 21 of the base 15 of the cartridge 5 in the close position of the cartridge 5. This arrangement results in a lifting mechanism lowering the IOL 1 from the level of the loading plane 16 to the level of the injection plane 17. In FIG. 9A, the cartridge 5 is open, when the winglets 12, 13 are apart from each other. The IOL 1 is loaded into the cartridge 5 by the loading plunge on the level of loading plane 16. Then the cartridge 5 is closed by approaching the winglets 12, 13 to each other as it is indicated by the arrows in FIG. 9A and 9B. In FIG. 9C, the cartridge 5 is closed and the IOL 1 is moved to the level of injection plane 17 by the winglets 12, 13. The lifting operation of the winglets 12, 13 is based on the interaction of the flanges 19 of the base 15 of the cartridge 5 with the recesses 18 of the winglets 12, 13, as well as on the interaction of the flanges 20 of the winglets 12, 13 at their bottom part with the recesses 21 of the base 15 of the cartridge 5. During the closing process the recesses 21 of the base 15 of the cartridge 5 make the winglets 12, 13 not just turn around but move downwards too. With this downward movement the inner space of the cartridge 5 with the IOL 1 is lifted from the level of loading plane 16 to the level of injection plane 17. During the closing process, the IOL 1 is distorted from a unfolded shape (IOL 1 in FIG. 9A) to a folded shape (IOL 1 in FIG. 9C). folded shape is necessary for safe injection through the nozzle tube to the patient eye.

In order to have a safe close position of the cartridge 5, the inner surface of one of the winglets 13 at its upper part can be provided with a rim 24 fitting to a groove 25 of the other winglet 12 at their upper part.

In and advantageous embodiment, the bottom parts of the winglets 12, 13 are provided with legs 29, 30 seating in the plane of the injection plunger 4 when the cartridge 5 is in open position. Legs 29, 30 getting contact to each other in the open position of the cartridge 5 prevent casual injection by blocking the injection plunger to move forward to the cartridge 5 as well as act as limiters for the winglets 12, 13 in the open position.

Operating Injectors for Non-Preloaded and Preloaded IOLs

In the next figures, the operating steps for non-preloaded and preloaded IOLs are described simultaneously in order to highlight the difference between the two methods.

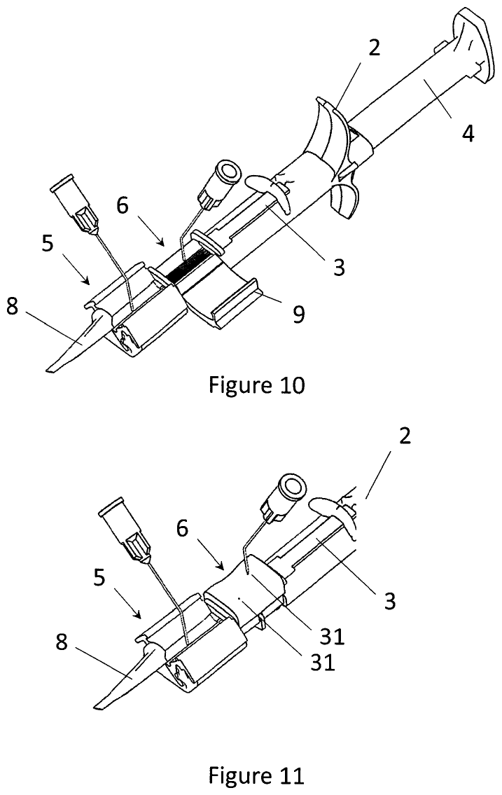

FIG. 10 shows the first step of operating an injector for injecting non-preloaded IOL comprising a cartridge 5 with a nozzle tube 8, a lens case 6, an injector body 2, an injection plunger 4 and a loading pusher 3. In this step, visco-elastic material is injected to the open cartridge 5 and to the open lens case 6. Visco-elastic material reduces the friction between the IOL 1 and the inner surface of the cartridge 5 and the lens case 6.

FIG. 11 shows the same step with an injector for injecting hydrophobic preloaded IOL. In this figure a part of the injector is illustrated only focusing to the difference to the previous case. In this case, the visco-elastic material is injected through holes 31 prepared on the top of the lens case 6 attached to the cartridge 5 with the nozzle tube 8.

In FIG. 12, the non-preloaded IOL 1 is placed to the lens case 6 having a lid 9 hinging on the lens case 6. Placing the IOL 1 into the lens case 6 requires no special exercise, since there is no need of pre-folding the IOL 1 at this stage. This step is missing in case of a hydrophobic preloaded IOL 1.

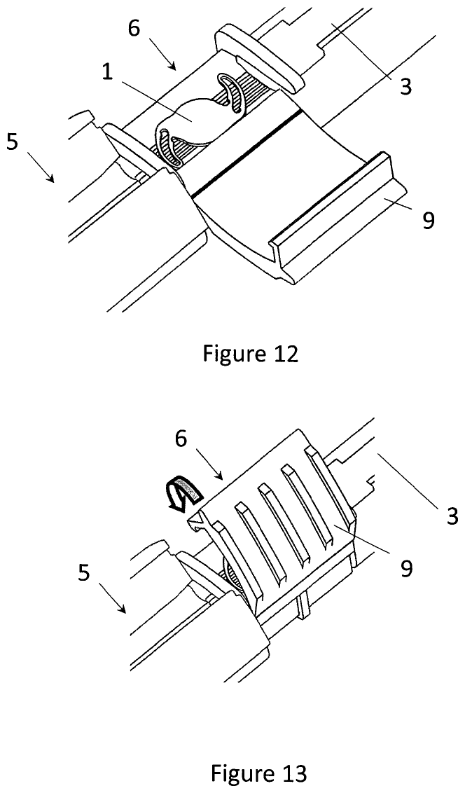

FIG. 13 illustrates the closing step of the lens case 6 for non-preloaded IOL 1. The lid 9 is closed up to the body of the lens case 6 as depicted by the arrow. By this step the IOL 1 is ready for loading.

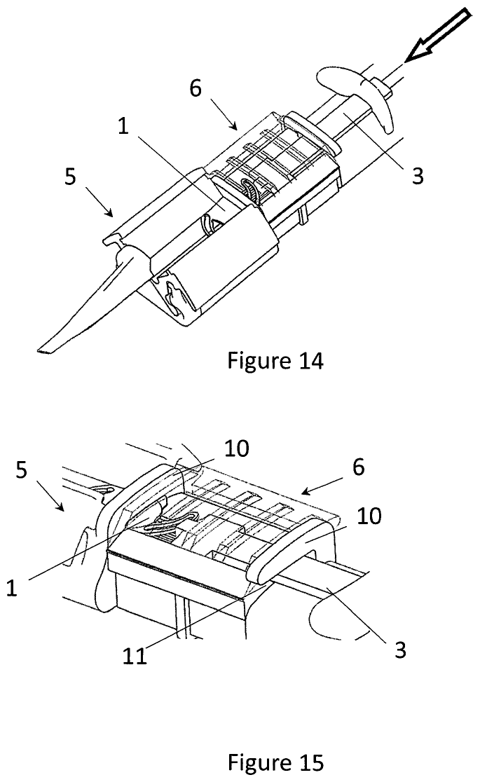

FIG. 14 shows the next step when the non-preloaded IOL 1, placed in the lens case 6, is moved to the cartridge 5 by pushing forward the loading pusher 3 as it is indicated by the arrow.

FIG. 15 shows the same step from another view. The loading pusher 3 is moving in the tunnel 11 below the gates 10 to the cartridge 5 pushing the IOL 1 from the lens case 6 to the cartridge 5.

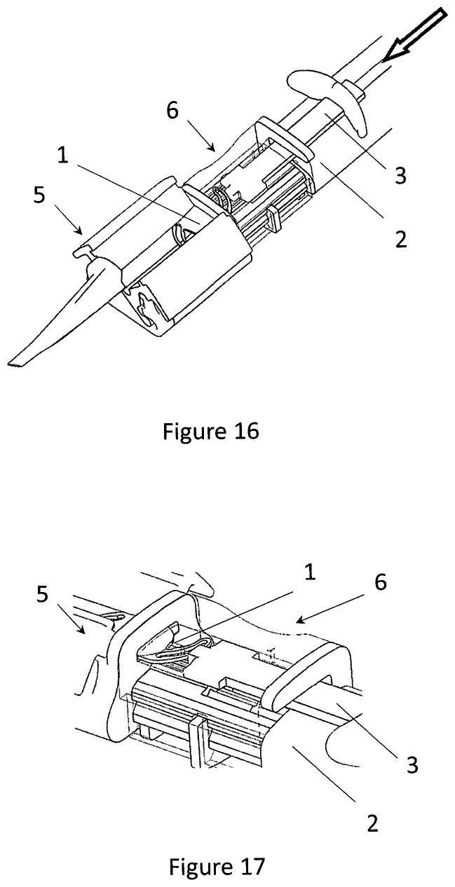

The same operation is carried out for the hydrophobic preloaded IOL 1 according to FIG. 16 and FIG. 17. In this phase, the IOL 1 is leaving the lens case 6 and reaching the cartridge 5 moved by the loading plunge 3.

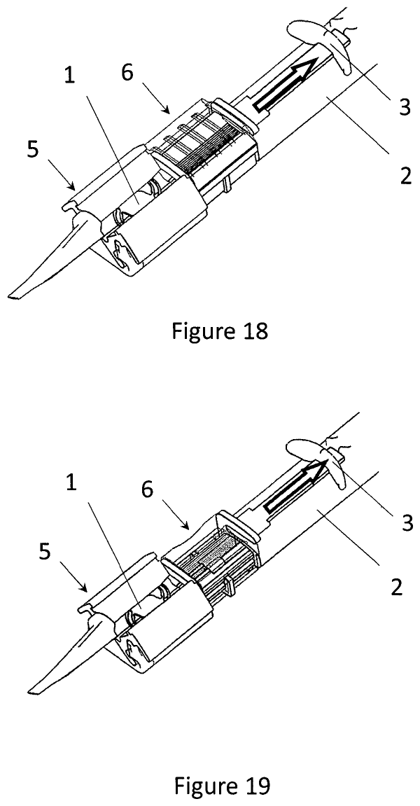

After the loading step, the loading pusher 3 is pulled back in case of non-preloaded as well as in preloaded IOL as it is shown in FIG. 18 and FIG. 19, respectively. Lens cases 6 are depicted transparently, to show that their inner space is empty.

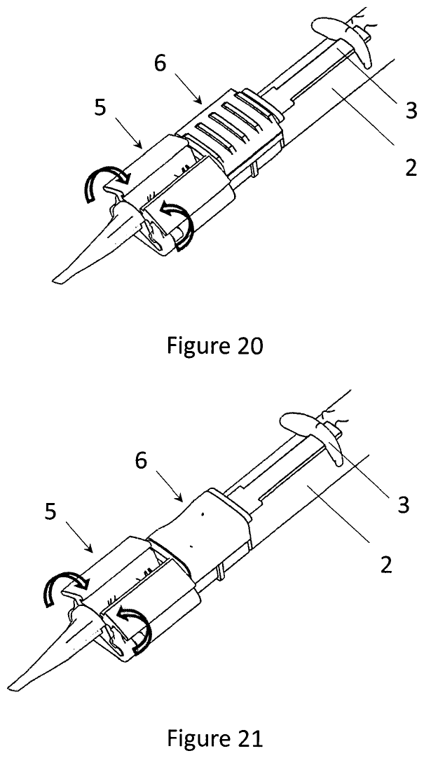

FIG. 20 shows the closing step of the cartridge 5 for non-preloaded and FIG. 21 for the hydrophobic preloaded IOL 1 when the IOL is lowered from the loading level to the injection level as described in details in FIGS. 9A-9C. The closing of the cartridge is indicated by arrows.

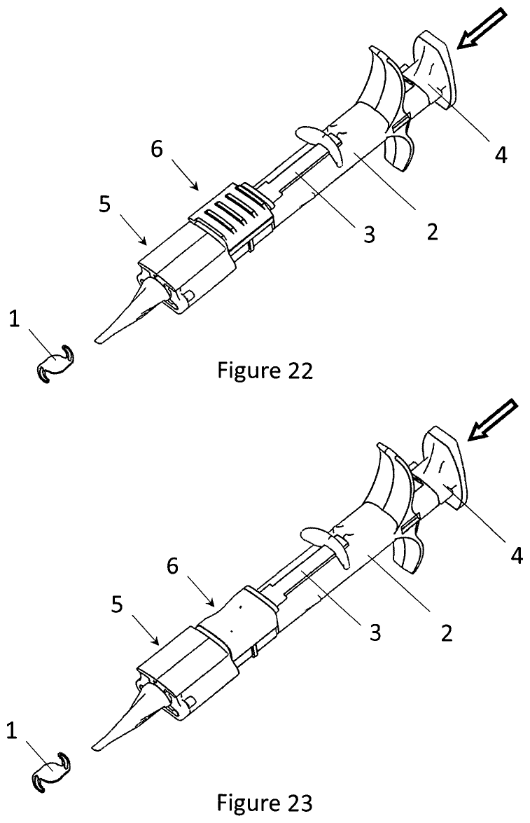

Reaching the injection level 17, the injection plunger 4 is pushed forward, injecting the IOL 1 to the patient eye. This step is also identical both for non-preloaded IOL as shown in FIG. 22, and for hydrophobic preloaded IOL 1 shown in FIG. 23.

In case of injecting a hydrophilic or hydrophobic IOL preloaded in a separate lens case 1, the method comprises the steps of a.) mounting the lens case 6 preloaded with a hydrophilic or hydrophobic IOL 1; b.) injecting visco-elastic material into the open cartridge 5 and into the lens case 6 through a hole 33; c.) moving the IOL 1 into the cartridge 5 by pushing forward the loading pusher 3; d.) pulling back the loading pusher 3; e.) closing the cartridge 5; f.) injecting the IOL 1 by pushing forward the injection plunger 4.

FIGS. 24, 25, 26 show the steps different from the operating steps of the hydrophobic preloaded IOL 1. FIG. 24 shows the docking step of the lens case, FIG. 25 shows the removal step of the stopper 34 from the lens case 6 and FIG. 26 shows the injection step of visco-elastic material into the lens case 6 through a hole 33 that accommodated the stopper 34.

Although three preferred embodiments of the present invention have been illustrated in the accompanying drawings and described in the foregoing detailed description, it is understood that the invention is not limited to the disclosed embodiment but is capable of numerous rearrangements, modifications, and substitutions, i.e. swop of recesses and protrusions on some parts of the injector without departing from the invention.

1. An injector for injecting an intraocular lens, IOL, (1) comprising
a cartridge (5) with a nozzle tube (8), a lens case (6), an injector body (2), an injection plunger (4) and a loading pusher (3),
wherein,
the injector body (2) accommodates the loading pusher (3) and the injection plunger (4) the axis of which are parallel to each other and are accommodated in the injector body (2) on a level of a loading (16) and on a level of an injection plane (17), respectively,
the cartridge (5) comprises two winglets (12, 13) connected to each other by a hinge joint (14) providing an open and a close position for the cartridge (5);
in the open position of the cartridge (5) the inner surface of the winglets (12, 13) forms an inner space on the level of the loading plane (16), where the axis of the inner space coincides with the axis of the loading pusher (3);
in the close position of the cartridge (5) the inner surface of the winglets (12, 13) forms an inner space on the level of the injection plane (17), where the axis of the inner space coincides with the axis of the injection plunger (4);
the lens case (6) is attached to the injector body (2) behind the cartridge (5), the inner surface of the lens case (6) forms an inner space for the unfolded IOL (1).

2. The injector for injecting IOL (1) of claim 1, wherein the outer surface of the winglets (12, 13) at their upper parts are provided with recesses (18) fitting to flanges (19) of the base (15) of the cartridge (5) in the open position of the cartridge (5), and are provided with flanges (20) at their bottom parts fitting to recesses (21) of the base (15) of the cartridge (5) in the close position of the cartridge (5).

3. The injector for injecting non-preloaded IOL (1) of claim 1, wherein the flange (20) of at least one winglet (12 or 13) is provided with a protrusion (22) fitting to a cavity (23) in the base (15) of the cartridge (5) in the close position of the cartridge (5).

4. The injector for injecting IOL (1) of claim 1, wherein the inner surface of one of the winglets (13) at its upper part is provided with a rim (24) fitting to a groove (25) of the other winglet (12) at their upper part.

5. The injector for injecting IOL (1) of claim 1, wherein the bottom parts of the winglets (12, 13) are provided with legs (29, 30) standing in the plane of the injection plunger (4) when the cartridge (5) is in open position.

6. The injector for injecting IOL (1) of claim 1, wherein the bottom of the lens case (6) is provided with rims (24) and the injector body (2) is provided with grooves (25), and the axis of inner space of the lens case (6) coincides with the axis of the loading pusher (3) when the rims (24) of the lens case (6) fit to the grooves (25) of the injector body (2).

7. The injector for injecting IOL (1) of claim 6, wherein the lens case (6) is provided with a lid (9) hinging on the lens case (6).

8. The injector for injecting IOL (1) of claim 1, wherein the lens case (6) is attached to the injector body (2) having an upper position where the axis of the inner space of the lens case (6) is above the level of the loading plane (16), and having a lower position where the axis of the inner space of the lens case (6) is on the level of the loading plane (16), where the axis of the inner space coincides with the axis of the loading pusher (3).

9. The injector for injecting IOL (1) of claim 8, wherein the injector body (2) is provided with gates (10) at both ends of the lens case (6) closing both end of the inner space of the lens case (6) in its upper position, and the injector body (2) is provided with tunnels (11) at both ends of the inner space of the lens case (6) in its lower position.

10. The injector for injecting IOL (1) of claim 8, wherein the bottom of the lens case (6) is provided with rims (26) fitting to upper grooves (27) of the injector body (2) in the upper position of the lens case (6) and fitting to lower grooves (28) in the lower position of the lens case (6).

11. A method for operating an injector for injecting non-preloaded intraocular lenses, IOL, (1) the injector comprising a cartridge (5) with a nozzle tube (8), a lens case (6), an injector body (2), an injection plunger (4) and a loading pusher (3), the method comprising the steps of
injecting visco-elastic material to the open cartridge and to the open lens case;
placing the IOL (1) to the lens case (6);
closing the lens case (6);
moving the IOL (1) to the cartridge (5) by pushing forward the loading pusher (3);
pulling back the loading pusher (3);
closing the cartridge (5);
injecting the IOL by pushing forward the injection plunger (4).

12. A method for operating an injector for injecting hydrophobic preloaded intraocular lenses, IOL, (1) the injector comprising a cartridge (5) with a nozzle tube (8), a lens case (6), an injector body (2), an injection plunger (4) and a loading pusher (3), the method comprising the steps of
moving the lens case (6) from an upper position to a lower position and injecting visco-elastic material to the open cartridge (5) and to the close lens case (6) through its holes (31);
moving the IOL (1) to the cartridge (5) by pushing forward the loading pusher (3);
closing the cartridge (5);
injecting the IOL (1) by pushing forward the injection plunger (4).

13. A method for operating an injector for injecting hydrophilic or hydrophobic intraocular lenses, IOL, (1) preloaded in a separate lens case, the injector comprising a cartridge (5) with a nozzle tube (8), a lens case (6), an injector body (2), an injection plunger (4) and a loading pusher (3), the method comprising the steps of
mounting the lens case (6) preloaded with a hydrophilic or hydrophobic IOL (1);
injecting visco-elastic material into the open cartridge (5) and into the lens case (6) through a hole (33);
moving the IOL into the cartridge (5) by pushing forward the loading pusher (3);
pulling back the loading pusher (3);
closing the cartridge (5);
injecting the IOL (1) by pushing forward the injection plunger (4).

14. The method of claim 13, wherein the step of mounting the lens case (6) comprises
docking the lens case (6) to the injector body (2), and
removing a stopper (34) from the lens case (6).
